(19) United States
(12) Patent Application Publication
(10) Pub. No.: US 2026/0202388 A1
(43) Pub. Date: Jul. 16, 2026

(54) METHOD OF EVALUATING ETCHANT GAS

(71) Applicant: Samsung Electronics Co., Ltd., Suwon-si (KR)

(72) Inventors: Thanh Cuong Nguyen, Suwon-si (KR); Heejeong Kim, Suwon-si (KR); Wonjoon Son, Suwon-si (KR); Seungmin Lee, Suwon-si (KR)

(21) Appl. No.: 19/296,455

(22) Filed: Aug. 11, 2025

(30) Foreign Application Priority Data
Jan. 16, 2025 (KR) 10-2025-0006829

(51) Int. Cl. G01N 33/00

(52) U.S. Cl. CPC G01N 33/0073

(57) ABSTRACT

A method of evaluating an etchant gas for selectively etching an target film includes obtaining input data of etchant gases, selecting a plurality of descriptors including a first descriptor, a second descriptor, and a third descriptor, the first descriptor determined by an activation energy of the target film, the second descriptor determined by a difference between an activation energy of a protection film and the activation energy of the target film, and the third descriptor determined by a binding energy of an intermediate product estimated to be generated during an etching process, calculating an etch rate of the target film, based on a first physical model obtained from the first and third descriptors and a temperature, calculating an etching selectivity of the target film with respect to the protection film, based on a second physical model obtained from the second and third descriptors and the etching temperature.

### CROSS-REFERENCE TO RELATED APPLICATION

This application claims priority under 35 U.S.C. § 119 to Korean Patent Application No. 10-2025-0006829, filed on Jan. 16, 2025, in the Korean Intellectual Property Office, the entirety of which is incorporated by reference herein.

### BACKGROUND

Due to the development of electronics technology, the downscaling of integrated circuit (IC) devices has rapidly progressed. In a process of manufacturing highly downscaled IC devices, etching and/or cleaning processes for selectively removing a film to be etched (hereinafter, referred to as an etching target film) may be performed while the etching target film is exposed on a wafer together with a film to be protected or a target film for protection (hereinafter, referred to as a protection target film) including a different material from the etching target film. Although wet etching and/or cleaning processes have relatively high isotropic etching effects, liquid etchants may affect areas other than an area to be etched.

### SUMMARY

Aspects of this disclosure provide methods of evaluating an etchant gas, by which an effective etchant gas that may be employed together with HF gas may be screened as an etchant gas for dry etching or dry cleaning an etching target film with a high selectivity while the etching target film is exposed on a wafer together with a protection target film.

According to some implementations of the present disclosure, there is provided a processor-implemented method of evaluating etchant gases for etching an etching target film while the etching target film and a protection target film are exposed to an etching atmosphere. The processor-implemented method includes obtaining input data of the etchant gases, estimating, based on the input data, a plurality of descriptors including a first descriptor, a second descriptor, and a third descriptor, the first descriptor being determined by an activation energy of the etching target film, the second descriptor being determined by a difference between an activation energy of the protection target film and the activation energy of the etching target film, and the third descriptor being determined by a binding energy of an intermediate product estimated to be generated during an etching process of etching the etching target film by using the etchant gases, calculating an etch rate of the etching target film when each of the etchant gases is used, based on a first physical model obtained from the first descriptor, the third descriptor, and data about an etching temperature, calculating an etching selectivity of the etching target film with respect to the protection target film when each of the etchant gases is used, based on a second physical model obtained from the second descriptor, the third descriptor, and the data about the etching temperature, and verifying the etchant gases, based on the etch rate and the etching selectivity.

According to some implementations of the present disclosure, there is provided a processor-implemented method of evaluating organic amine gases for etching an etching target film by using an etchant gas comprising a mixture of hydrogen fluoride (HF) gas and an organic amine gas while the etching target film and a protection target film are exposed to an etching atmosphere. The processor-implemented method includes obtaining input data of organic amine gases, estimating, based on the input data, a plurality of descriptors including a first descriptor, a second descriptor, and a third descriptor, the first descriptor being determined by an activation energy of the etching target film, the second descriptor being determined by a difference between an activation energy of the protection target film and the activation energy of the etching target film, and the third descriptor being determined by a binding energy of an organic ammonium salt containing fluorine atoms, which is estimated to be generated during an etching process of etching the etching target film by using the organic amine gases, calculating an etch rate of the etching target film when each of the organic amine gases is used, based on a first physical model obtained from the first descriptor, the third descriptor, and data about an etching temperature, calculating an etching selectivity of the etching target film with respect to the protection target film when each of the organic amine gases is used, based on a second physical model obtained from the second descriptor, the third descriptor, and the data about the etching temperature, and verifying the organic amine gases based on the etch rate and the etching selectivity.

According to some implementations of the present disclosure, there is provided a method of evaluating organic amine gases for etching a silicon oxide film in a non-plasma manner by using an etchant gas comprising a mixture of HF gas and an organic amine gas while the silicon oxide film and a silicon nitride film are exposed to an etching atmosphere. The method of evaluating organic amine gases includes obtaining input data of organic amine gases, predicting a reaction path on a surface of each of the silicon oxide film and the silicon nitride film, calculating a first fluorination reaction activation energy on the surface of the silicon oxide film, calculating a second fluorination reaction activation energy on the surface of the silicon nitride film, calculating a difference between the second fluorination reaction activation energy and the first fluorination reaction activation energy, calculating a binding energy of an organic ammonium fluorosilicate salt estimated to be generated during an etching process of etching the silicon oxide film by using the organic amine gas, calculating an etch rate of the silicon oxide film when each of the organic amine gases is used, based on a first physical model obtained from the first fluorination reaction activation energy and the binding energy of the organic ammonium fluorosilicate salt, calculating an etching selectivity of the silicon oxide film with respect to the silicon nitride film when each of the organic amine gases is used, based on a second physical model obtained from a difference between the second fluorination reaction activation energy and the first fluorination reaction activation energy and the binding energy of the organic ammonium fluorosilicate salt, and verifying the organic amine gases, based on the etch rate of the silicon oxide film and the etching selectivity of the silicon oxide film with respect to the silicon nitride film.

According to some implementations of the present disclosure, there is provided an etchant gas evaluation device may include at least one processor and a storage medium configured to store instructions to perform a method of evaluating an etchant gas by using the at least one processor. The method of evaluating the etchant gas is used to determine an organic amine gas to be employed in an etching process of selectively dry etching an etching target film by using an etchant gas including a mixture of HF gas and the organic amine gas while the etching target film and a protection target film are exposed together to an etching atmosphere. The method of evaluating the etchant gas includes obtaining input data of the etchant gases, determining, based on the input data, a plurality of descriptors including a first descriptor, a second descriptor, and a third descriptor, the first descriptor being determined by an activation energy of the etching target film, the second descriptor being determined by a difference between an activation energy of the protection target film and the activation energy of the etching target film, and the third descriptor being determined by a binding energy of an intermediate product estimated to be generated during an etching process of etching the etching target film by using the etchant gases, calculating an etch rate of the etching target film when each of the etchant gases is used, based on a first physical model obtained from the first descriptor, the third descriptor, and data about an etching temperature, calculating an etching selectivity of the etching target film with respect to the protection target film when each of the etchant gases is used, based on a second physical model obtained from the second descriptor, the third descriptor, and the data about the etching temperature, and verifying the etchant gases, based on the etch rate and the etching selectivity.

### BRIEF DESCRIPTION OF THE DRAWINGS

FIG. 1 is a block diagram of an example of an etchant gas evaluation device;

FIG. 2 is a flowchart illustrating an example of a method of evaluating an etchant gas;

FIG. 3 is a diagram illustrating an example of a mechanism in which a silicon oxide film and a silicon nitride film are etched by using hydrogen fluoride (HF) gas and an organic amine gas;

FIGS. 4A, 4B, 4C, and 4D are graphs illustrating examples of a physical model including first to third descriptors based on an etching mechanism that is used in a method of evaluating an etchant gas;

FIG. 5 is a flowchart illustrating an example of a method of evaluating an etchant gas;

FIG. 6A is a table showing examples of organic amines included in a database of an etchant gas evaluation device;

FIG. 6B is a table showing examples of character strings in a one-dimensional format for organic amines that are screened by using a method of evaluating an etchant gas;

FIG. 7 is a flowchart illustrating an example of estimating descriptors in a method of evaluating an etchant gas;

FIG. 8 is a graph illustrating an energy variation with respect to a reaction path of a silicon oxide film or a silicon nitride film when the silicon oxide film or the silicon nitride film is exposed to an etchant gas mixture of HF gas and an organic amine gas;

FIG. 9 is a flowchart illustrating an example of estimating descriptors according to process P20 of FIG. 5;

FIG. 10 is a flowchart illustrating a process of calculating an etch rate of a silicon oxide film, based on a physical model A;

FIG. 11 is a flowchart illustrating a process of calculating an etching selectivity of a silicon oxide film with respect to a silicon nitride film, in a method of evaluating an etchant gas;

FIG. 12 is a flowchart illustrating an example of a process of verifying organic amines to be evaluated, in a method of evaluating an etchant gas; and

FIGS. 13A, 13B, 13C, and 13D are graphs showing examples of a process of verifying organic amines to be evaluated, by using a physical model including descriptors based on an etching mechanism.

### DETAILED DESCRIPTION

Hereinafter, examples will be described in detail with reference to the accompanying drawings. The same reference numerals are used to denote the same elements in the drawings, and repeated descriptions thereof will be omitted.

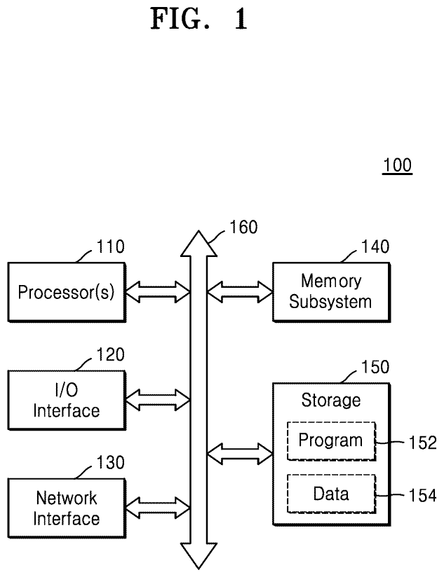

FIG. 1 is a block diagram of an etchant gas evaluation device 100. Methods of evaluating etchant gases, as described below, may be performed by using the etchant gas evaluation device 100 described with reference to FIG. 1.

Referring to FIG. 1, the etchant gas evaluation device 100 may include a stationary computing system (e.g., a desktop computer, a workstation, and a server) or include a portable computing system (e.g., a laptop computer).

The etchant gas evaluation device 100 may include at least one processor 110, an input/output (I/O) interface 120, a network interface 130, a memory subsystem 140, a storage 150, and a bus 160. The at least one processor 110, the I/O interface 120, the network interface 130, the memory subsystem 140, and the storage 150 may communicate with each other via the bus 160.

The at least one processor 110 may be referred to as at least one processing unit and may execute a program together with, for example, a central processing unit (CPU), a graphics processing unit (GPU), a neutral processing unit (NPU), and a digital signal processor (DSP). For example, the at least one processor 110 may access the memory subsystem 140 via the bus 160 and execute instructions stored in the memory subsystem 140. In some implementations, the etchant gas evaluation device 100 may further include an accelerator, which is a dedicated hardware designed to perform a specific function at high speed.

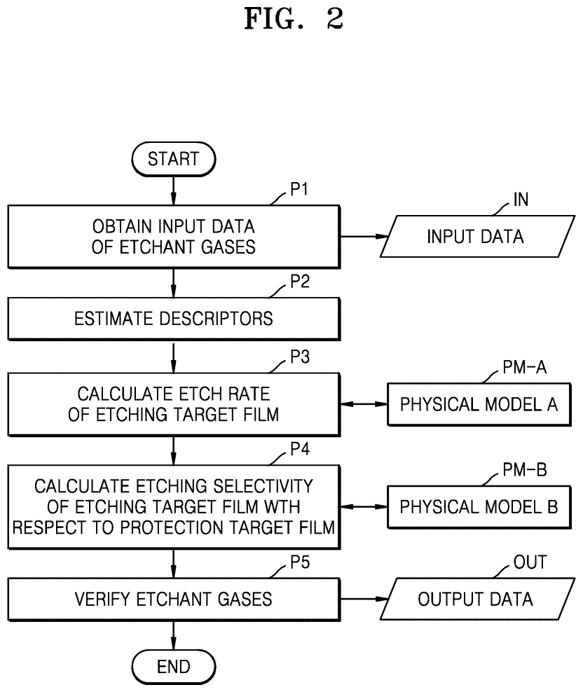

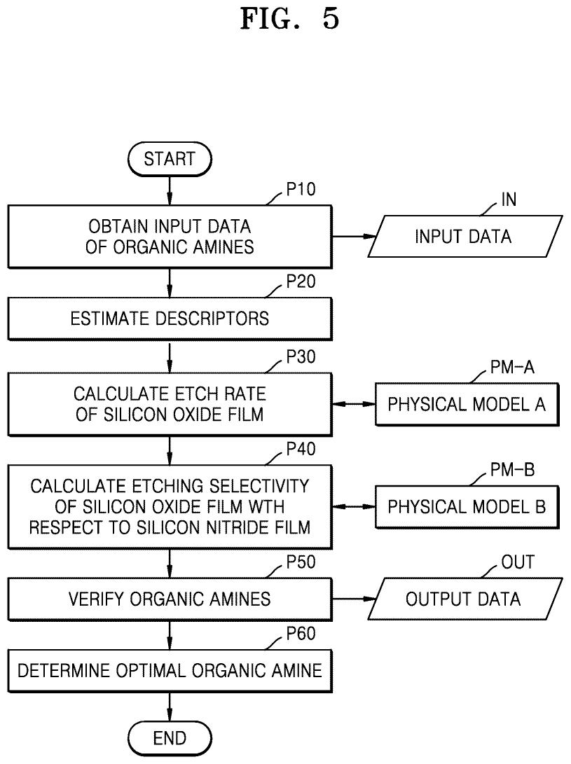

The I/O interface 120 may include an input device (e.g., a keyboard and a pointing device) and/or an output device (e.g., a display device and a printer) or provide access to the input device and/or the output device. A user may trigger the execution of a program 152 and/or the loading of data 154 via the I/O interface 120 or may input input data IN described below with reference to FIGS. 2 and 5. In some implementations, the user may input, via the I/O interface 120, input data IN of etchant gases used in process P1 of a method of evaluating an etchant gas, which is described below with reference to FIG. 2, or input data IN of organic amines used in process P10 of a method of evaluating an etchant gas, which is described below with reference to FIG. 5, into the program 152 of the storage 150. As another example, the etchant gas evaluation device 100 may output, as output data OUTPUT, an etchant gas verified in process P5 of the method of evaluating the etchant gas, which is described below with reference to FIG. 2, or organic amines verified in process P50 of the method of evaluating the etchant gas, which is described below with reference to FIG. 5.

The network interface 130 may provide access to a network provided outside the etchant gas evaluation device 100. For example, the network may include a plurality of computing systems and communication links, and the communication links may include wired links, optical links, wireless links, or links of any other type.

The memory subsystem 140 may store the program 152 used to perform the method of evaluating the etchant gas, or at least a portion of the program 152, and the at least one processor 110 may perform at least some of operations included in the method of evaluating the etchant gas by executing a program (or instructions) stored in the memory subsystem 140. In some implementations, the memory subsystem 140 may include at least one storage medium selected from flash memory type, hard disk type, multimedia card micro type, card-type memory, random access memory (RAM), static RAM (SRAM), read-only memory (ROM), electrically erasable programmable ROM (EEPROM), programmable ROM (PROM), magnetic memory, magnetic disk, and optical disk.

The storage 150 may be a non-transitory storage medium, and thus, data stored in the storage 150 may not be lost even when power supplied to the etchant gas evaluation device 100 is cut off. For example, the storage 150 may include a non-volatile memory device or include a storage medium, such as magnetic tape, optical disk, and magnetic disk. In addition, the storage 150 may be removably connected to the etchant gas evaluation device 100.

The program 152 and data 154 may be stored in the storage 150. Before a process of evaluating the etchant gas is executed by the at least one processor 110, at least a portion of the program 152 may be loaded into the memory subsystem 140. In some implementations, the storage 150 may store a file written in a program language, and the program 152 generated by a complier from the file or at least a portion of the program may be loaded into the memory subsystem 140. The at least one processor 110 may perform at least part of the method of evaluating the etchant gas by executing the program 152. The data 154 stored in the storage 150 may include data used to perform a method of evaluating an etchant gas for example, input data IN, a physical model A (PM-A), a physical model B (PM-B), or a combination thereof, which are described below with reference to FIG. 2 or FIG. 5. In addition, the data 154 stored in the storage 150 may include data generated by performing the method of evaluating the etchant gas, for example, output data OUT described below with reference to FIG. 2 or FIG. 5.

FIG. 2 is a flowchart of an example of a method of evaluating an etchant gas. In this example, the method is a method of screening an effective etchant gas, which may be employed to remove an etching target film with a high selectivity by using a dry etching process in a non-plasma manner, while the etching target film is exposed on a wafer together with a protection target film.

In some implementations, the etching target film includes a silicon oxide film (e.g., a SiO₂ film). As used herein, the term “silicon oxide film” may refer to silicon oxide films commonly used in the art, for example, a film including at least one selected from a spin-on-dielectric (SOD) film, a high-density plasma (HDP) film, a thermal oxide film, a borophosposilicate glass (BPSG) film, a phosphosilicate glass (PSG) film, a borosilicate glass (BSG) film, a polysilazane (PSZ) film, a fluorinated silicate glass (FSG) film, a low-pressure tetraethyl ortho silicate (LP-TEOS) film, a plasma enhanced tetraethyl ortho silicate (PETEOS) film, a high-temperature oxide (HTO) film, a medium-temperature oxide (MTO) film, an undoped silicate glass (USG) film, a spin-on glass (SOG) film, an advanced planarization layer (APL) film, an atomic layer deposition (ALD) film, a plasma enhanced (PE)-oxide film, or an O₃-tetraethyl ortho silicate (O₃-TEOS) film, without being limited thereto.

The protection target film includes a film for which there is an etching selectivity for the etching target film compared to the protection target film. In some implementations, the protection target film includes a film including silicon atoms and nitrogen atoms. For example, the protection target film may be referred to as a SiNₓ film or a silicon nitride film. As used herein, the so-called SiNₓ film or silicon nitride film may be interpreted as a film including silicon atoms and nitrogen atoms. For example, the protection target film may include Si₃N₄, SiON, SiOCN, SiCN, or a combination thereof, without being limited thereto. Unless described otherwise, the term “etching” is used herein to encompass both etching processes and cleaning processes.

In process P1 of FIG. 2, input data of etchant gases may be obtained from a database.

In some implementations, the etchant gases may be selected from organic amines. In some implementations, the etchant gases may be selected from alcohols.

In some implementations, to obtain data about etchant gases in process P1 of FIG. 2, at least some etchant gases selected from etchant gases included in a database of data 154 may be screened in the etchant gas evaluation device 100 illustrated in FIG. 1. In the etchant gas evaluation device 100 illustrated in FIG. 1, the data 154 stored in the storage 150 may include a database of various etchant gases, for example, organic amines having various structures or alcohols having various structures.

The etchant gases obtained in process P1 of FIG. 2 may be represented as character strings in a simple one-dimensional format. The character strings in the one-dimensional format may be represented as a Simplified Molecular-input Line-entry System (SMILES) code, a Smiles Arbitrary Target Specification (SMARTS) code, an International Chemical Identifier (InChi) code, or the like.

In process P2 of FIG. 2, a plurality of descriptors may be estimated. The plurality of descriptors may include descriptors based on an etching mechanism. For example, the plurality of descriptors may include a first descriptor determined by an activation energy of the etching target film, a second descriptor determined by a difference between an activation energy of the protection target film and the activation energy of the etching target film, and a third descriptor determined by a binding energy of an intermediate product that is estimated to be generated during an etching process of etching the etching target film by using the etchant gases. In some implementations, the intermediate product may be estimated based on first-principles simulations. Detailed description and examples of the first to third descriptors are provided below with reference to FIGS. 5 and 7 to 9.

In process P3 of FIG. 2, an etch rate of the etching target film may be calculated. The process of calculating the etch rate of the etching target film according to process P3 of FIG. 2 may be performed, based on a physical model A (PM-A) based on the etching mechanism. The physical model A (PM-A) may be obtained from the first descriptor, the third descriptor, and data about an etching temperature.

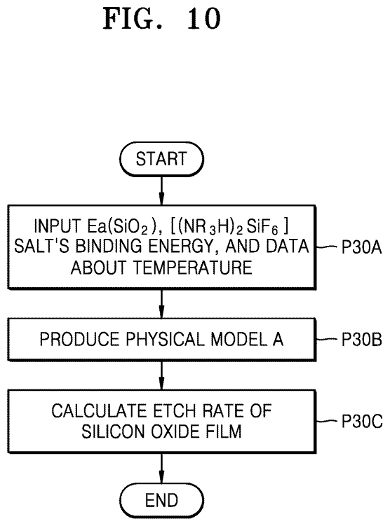

Detailed description and examples of the process of calculating the etch rate of the etching target film according to process P3 of FIG. 2, based on the physical model A (PM-A), are provided below with reference to FIG. 10. As used herein, the physical model A may be referred to as a first physical model.

In process P4 of FIG. 2, an etching selectivity of the etching target film with respect to the protection target film may be calculated. The process of calculating the etching selectivity of the etching target film with respect to the protection target film according to process P4 of FIG. 2 may be performed, based on a physical model B (PM-B) based on the etching mechanism. The physical model B (PM-B) may be obtained from the second descriptor, the third descriptor, and data about the etching temperature.

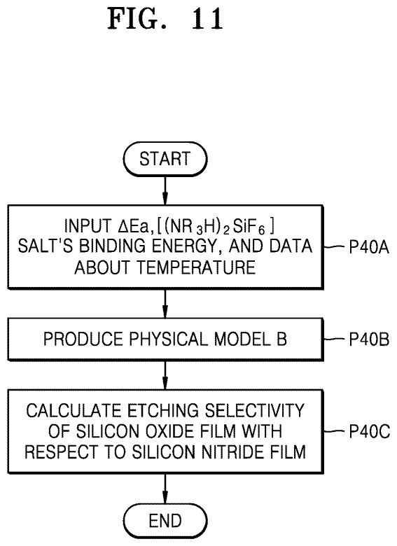

Detailed description and examples of the process of calculating the etching selectivity of the etching target film with respect to the protection target film according to process P4 of FIG. 2, based on the physical model B (PM-B), are provided below with reference to FIG. 11. As used herein, the physical model B may be referred to as a second physical model.

In process P5 of FIG. 2, etchant gases to be evaluated may be verified. The verification of the etchant gases to be evaluated may be performed based on the etch rate of the etching target film, which is calculated in process P3 of FIG. 2, and the etching selectivity of the etching target film with respect to the protection target film, which is calculated in process P4 of FIG. 2.

In some implementations, to verify the etchant gases to be evaluated, a first threshold value of an allowable etch rate of the etching target film and a second threshold value of an allowable etching selectivity of the etching target film with respect to the protection target film may be set. Thereafter, a target etchant gas corresponding to a case in which the etch rate of the etching target film is higher than or equal to the first threshold value and a case in which the etching selectivity of the etching target film with respect to the protection target film is higher than or equal to the second threshold value may be determined, or selected, as an optimal etchant gas.

In an example of a process of manufacturing an integrated circuit (IC) device, to remove the silicon oxide film, which is the etching target film, with a high selectivity while the silicon oxide film is exposed on the wafer together with the silicon nitride film, which is protection target film, a process of dry etching or dry cleaning the silicon oxide film in a non-plasma manner by using an etchant gas mixture including hydrogen fluoride (HF) gas and NH₃ gas may be used. However, to further increase an etch rate of the silicon oxide film compared to a case in which the etchant gas mixture including the HF gas and the NH₃ gas is used, a process of dry etching or dry cleaning the silicon oxide film in a non-plasma manner by using an etchant gas mixture including HF gas and an organic amine gas may be used. To this end, it may be advantageous to screen and design a relatively efficient target organic amine, from among numerous organic amine molecules that may be employed together with HF gas.

To solve the technical objectives described above, some aspects of this disclosure provide methods of evaluating an etchant gas, which may screen an effective etchant gas by calculating an etch rate of a silicon oxide film and an etching selectivity of the silicon oxide film with respect to the silicon nitride film when each of etchant gases to be evaluated (e.g., organic amines) is employed by using physical models based on the etching mechanism.

In the method of evaluating the etchant gas, according to some implementations, to identify a target organic amine capable of providing optimal selective etching effects during the etching process of dry etching the silicon oxide film in the non-plasma manner by using the etchant gas mixture including the HF gas and the organic amine gas, a descriptor determined by “a fluorination reaction activation energy difference,” which is calculated based on the first-principles simulations, and a descriptor determined by “a binding energy of an organic ammonium fluorosilicate salt,” which is an intermediate product containing fluorine atoms estimated to be generated during a reaction path of the etching process, may be used. From among the organic amines evaluated by using the method described above, an organic amine that exhibits a relatively high etch rate of the silicon oxide film and a relatively high etching selectivity of the silicon oxide film with respect to a silicon nitride film may be determined as an efficient target organic amine.

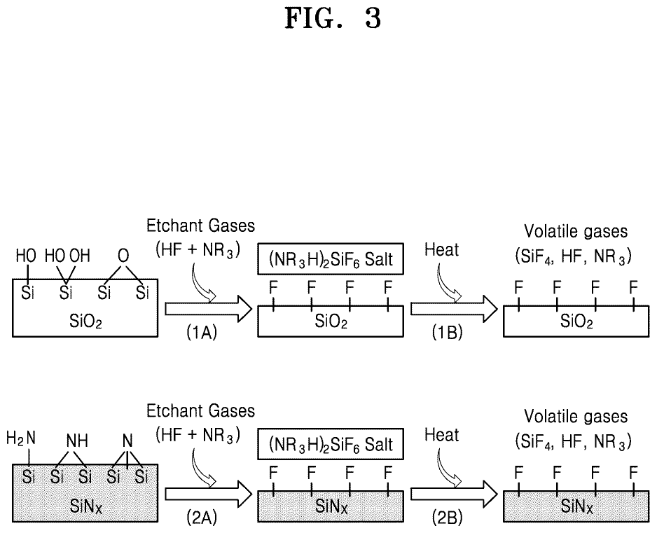

FIG. 3 is a diagram illustrating an example of a mechanism in which a silicon oxide film and a silicon nitride film are etched by using HF gas and an organic amine gas.

Main operations in a mechanism in which a silicon oxide film and a silicon nitride film are etched by using an etchant gas mixture including HF gas and an organic amine gas may include a first operation and a second operation as follows.
(1) The first operation is an operation indicated by 1A and 2A in FIG. 3, in which a portion of the silicon oxide film or the silicon nitride film may be chemically removed by forming a SiF₄ by-product on a surface of the silicon oxide film or the silicon nitride film due to a fluorination reaction between an etchant gas mixture including HF gas and an organic amine gas (NR₃, where R is an organic group) and the silicon oxide film or the silicon nitride film. In the first operation, the organic amine gas may act as a catalyst or inhibitor having an activation energy Ea for promoting or inhibiting the fluorination reaction. The SiF₄ by-product may easily react with HF and NR₃ to form an organic ammonium fluorosilicate [(NR₃H)₂SiF₆] salt. The reaction in the first operation may be shown as in Reaction scheme 1:

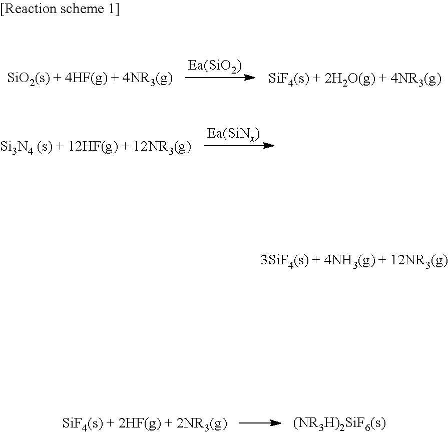

(2) The second operation may be an operation indicated by 1B and 2B in FIG. 3, in which the [(NR₃H)₂SiF₆] salt may decompose due to a subsequent thermal process. The reaction in the second operation may be shown in Reaction scheme 2:

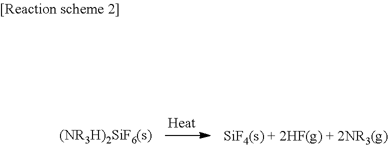

The performance of the etchant gas mixture including the HF gas and the organic amine gas may be evaluated based on an etch rate of each of the silicon oxide film and the silicon nitride film and an etching selectivity of the silicon oxide film with respect to the silicon nitride film. The etch rate of each of the silicon oxide film and the silicon nitride film may be affected by at least the following two factors. A first factor is a fluorination reaction activation energy Ea(SiO₂) of the silicon oxide film and a fluorination reaction activation energy Ea(SiNₓ) of the silicon nitride film, and a second factor is decomposability of the [(NR₃H)₂SiF₆] salt. Accordingly, a selective etching effect of the silicon oxide film may be improved by selecting an organic amine exhibiting a relatively small Ea(SiO₂) value and relatively low [(NR₃H)₂SiF₆] salt's binding energy (i.e., the decomposability of the [(NR₃H)₂SiF₆] salt).

In addition, the etching selectivity of the silicon oxide film with respect to the silicon nitride film may depend on a difference ΔEa [ΔEa=Ea(SiNₓ)−Ea(SiO₂)] between Ea(SiNₓ) and Ea(SiO₂). Herein, as a ΔEa value becomes greater, the etching selectivity of the silicon oxide film with respect to the silicon nitride film may become higher. Therefore, efficient organic amine may be screened by using the etch rate of the silicon oxide film and the etching selectivity of the silicon oxide film with respect to the silicon nitride film as estimation criteria.

When various organic amines are used together with HF gas to dry etch the silicon oxide film with a high selectivity in a non-plasma manner while the silicon oxide film is exposed to an etching atmosphere together with the silicon nitride film, a physical model including a plurality of different descriptors based on the etching mechanism may be used to calculate the etch rate of the silicon oxide film and the etching selectivity of the silicon oxide film with respect to the silicon nitride film. The plurality of descriptors based on the etching mechanism may include (1) a first descriptor determined by the fluorination reaction activation energy [Ea(SiO₂)] of the silicon oxide film, (2) a second descriptor determined by a difference (ΔEa) [ΔEa=Ea(SiNₓ)−Ea(SiO₂)] between the fluorination reaction activation energy Ea(SiNₓ) of the silicon nitride film and the fluorination reaction activation energy [Ea(SiO₂)] of the silicon oxide film, and (3) a third descriptor determined by a [(NR₃H)₂SiF₆] salt's binding energy. In some implementations, the [(NR₃H)₂SiF₆] salt's binding energy may be a value estimated based on first-principles simulations.

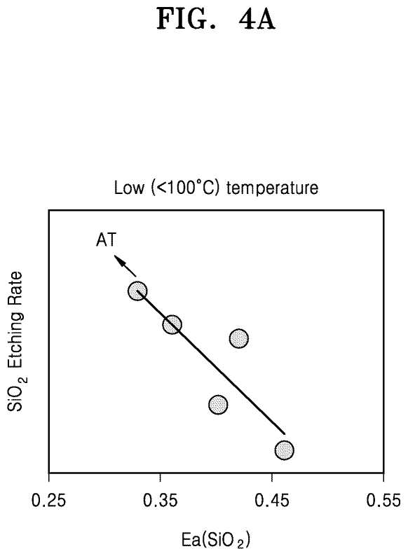

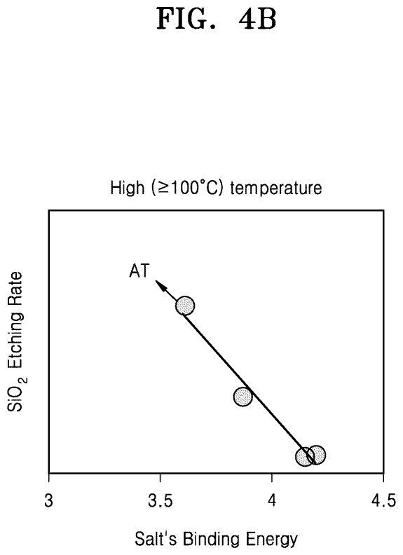

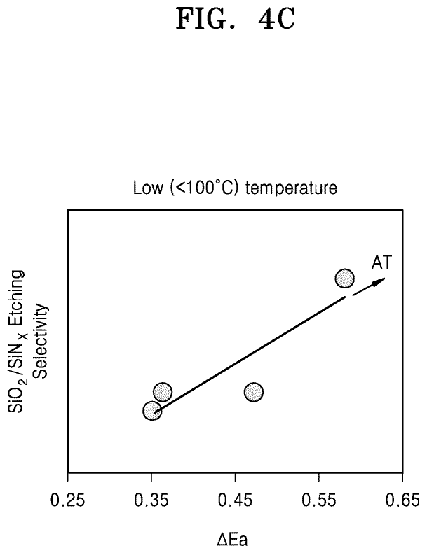

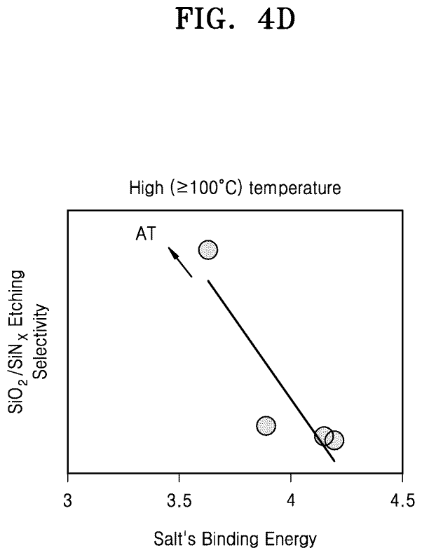

FIGS. 4A, 4B, 4C, and 4D are graphs illustrating examples of a physical model including first to third descriptors based on the etching mechanism described above. In each of FIGS. 4A, 4B, 4C, and 4D, the unit of each of a horizontal axis and a vertical axis may be an arbitrary unit.

Referring to FIG. 4A, when an etching temperature is below 100° C. (e.g., 60° C.), fluorination reaction activation energy Ea(SiO₂) of a silicon oxide film (SiO₂ film) may be inversely proportional to an etch rate of the silicon oxide film. From among the evaluated organic amines, an organic amine having a lower Ea(SiO₂) value and a higher etch rate of the silicon oxide film (SiO₂ film) may be evaluated to be a more efficient organic amine (e.g., a target organic amine AT).

Referring to FIG. 4B, when an etching temperature is 100° C. or higher (e.g., 120° C.), a [(NR₃H)₂SiF₆] salt's binding energy may be inversely proportional to an etch rate of a silicon oxide (SiO₂) film. From among the evaluated organic amines, an organic amine having a lower binding energy of the [(NR₃H)₂SiF₆] salt and a higher etch rate of the silicon oxide film (SiO₂ film) may be evaluated to be a more efficient organic amine (e.g., a target organic amine AT).

Referring to FIG. 4C, when an etching temperature is below 100° C. (e.g., 60° C.), a difference ΔEa between Ea(SiNₓ) and Ea(SiO₂) may be proportional to an etching selectivity of a silicon oxide film with respect to a silicon nitride film. From among the evaluated organic amines, an organic amine having a higher ΔEa value and a higher etching selectivity of the silicon oxide film with respect to the silicon nitride film may be evaluated to be a more efficient organic amine (e.g., a target organic amine AT).

Referring to FIG. 4D, when an etching temperature is 100° C. or higher (e.g., 120° C.), a [(NR₃H)₂SiF₆] salt's binding energy may be inversely proportional to an etching selectivity of a silicon oxide film with respect to a silicon nitride film. From among the evaluated organic amines, an organic amine having a lower [(NR₃H)₂SiF₆] salt's binding energy and a higher etching selectivity of the silicon oxide film with respect to the silicon nitride film may be evaluated to be a more efficient organic amine (e.g., a target organic amine AT).

In a method of evaluating an etchant gas, in some implementations, the following operations may be performed. In some implementations, the following operations described below may be performed by using a program 152 stored in the storage 150 of the etchant gas evaluation device 100 illustrated in FIG. 1.
(1) During or for a process of dry etching a silicon oxide film with a high selectivity in a non-plasma manner while the silicon oxide film is exposed to an etching atmosphere together with a silicon nitride film, an etch rate of a silicon oxide film obtained from organic amines to be evaluated and an etching selectivity of the silicon oxide film with respect to the silicon nitride film may be calculated by using a plurality of descriptors, which are estimated from a fluorination reaction and a [(NR₃H)₂SiF₆] salt's binding energy, based on first-principles simulations. The plurality of descriptors may include a physical model A and a physical model B, based on a fluorination reaction activation energy Ea(SiO₂) of a silicon oxide film, a difference ΔEa [ΔEa=Ea(SiNₓ)−Ea(SiO₂)] in fluorination reaction activation energy, and the [(NR₃H)₂SiF₆] salt's binding energy.
(2) From a database of organic amine molecules, an organic amine suitable for an etchant gas mixture including “HF+organic amine” may be verified by comparing respective values of the etch rate of the silicon oxide film and respective values of the etching selectivity of the silicon oxide film with respect to the silicon nitride film.

Accordingly, the etch rate of the silicon oxide film obtained from organic amines, which may be used in a dry etching process or dry cleaning process performed in a non-plasma manner, and the etching selectivity of the silicon oxide film with respect to the silicon nitride film may be rapidly calculated. Therefore, in a process of manufacturing an IC device (e.g., a semiconductor logic device or a semiconductor memory device), an etchant gas mixture including an optimal combination of “HF+organic amine” may be selected and used in various processes that use a process of selectively removing only a silicon oxide film, from among the silicon oxide film and a silicon nitride film, for example, a process of plasma etching a self-aligned contact, a process of forming an air gap, or a process of selectively etching only a silicon oxide film, from among a silicon nitride film and the silicon oxide film, in a charge trap layer included in a vertical NAND (VNAND) device. For example, the methods of evaluating etchant gases described herein may provide a useful strategy for developing a new material layer and/or pattern structure to improve the performance of an IC device manufacturing process.

FIG. 5 is a flowchart illustrating an example of a method of evaluating an etchant gas. FIG. 5 illustrates an example of a method of screening an effective organic amine, which may be employed when an etchant gas mixture including “HF+organic amine” is used as an etchant gas for removing a silicon oxide film formed on a wafer with a high selectivity by using a dry etching process in a non-plasma manner, while the silicon oxide film is exposed to an etching atmosphere together with another material (e.g., a silicon nitride film).

In process P10 of FIG. 5, input data of organic amines may be obtained from a database of organic amines.

In some implementations, to obtain data of organic amines in process P10 of FIG. 5, at least some organic amines may be screened from organic amines included in a database of data 154 in the etchant gas evaluation device 100 illustrated in FIG. 1.

In the etchant gas evaluation device 100 illustrated in FIG. 1, the data 154 stored in the storage 150 may include a database of various organic amines. In the database, the organic amines may include aliphatic amine, aromatic amine, and heterocyclic amine. In addition, the organic amines may be classified into primary amines, secondary amines, and tertiary amines depending on the number of hydrogen atoms bonded to nitrogen atoms. The database included in the data 154 of the storage 150 may include various types of organic amines.

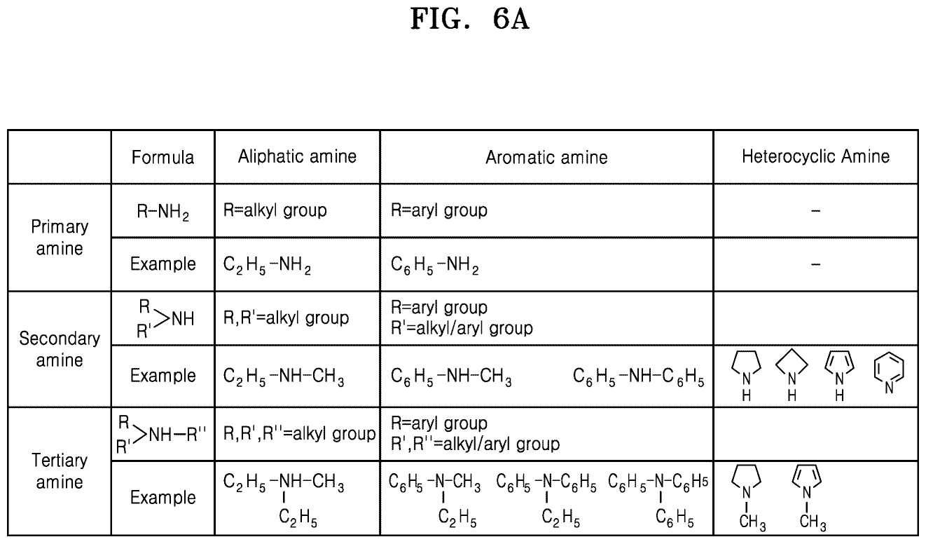

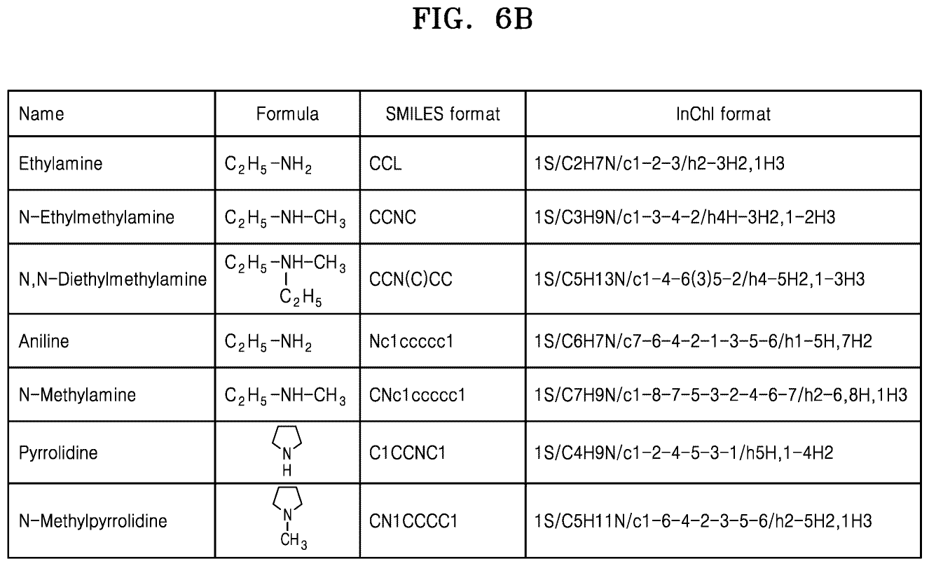

FIG. 6A is a table showing some organic amines that may be included in a database. In some implementations, to obtain data about organic amines in process P10 of FIG. 5, at least some of the organic amines illustrated in FIG. 6A may be screened from organic amines included in a database of data 154 of the etchant gas evaluation device 100 illustrated in FIG. 1.

In some implementations, the organic amines screened in process P10 of FIG. 5 may include aliphatic amine, aromatic amine, and heterocyclic amine as illustrated in FIG. 6A. In addition, the screened organic amines may be classified into primary amine, secondary amine, and tertiary amine depending on the number of hydrogen atoms bonded to nitrogen atoms.

As illustrated in FIG. 6A, the primary amine may be represented by a chemical formula of R—NH₂, wherein R denotes a C1 to C30 alkyl group or a C1 to C30 aryl group. For example, the primary amine may include C₂H₅—NH₂ or C₆H₅—NH₂, without being limited thereto. The secondary amine may be represented by a chemical formula of R—NH—R′, wherein each of R and R′ denotes a C1 to C30 alkyl group or a C1 to C30 aryl group. R and R′ may be mutually bonded to each other to form a ring with nitrogen atoms (N). For example, the secondary amine may include C₂H₅—NH—CH₃, C₆H₅—NH—CH₃, C₆H₅—NH—C₆H₅, pyrrolidine, azetidine, pyrrole, pyridine, and/or the like, without being limited thereto.

In some implementations, an etchant gas including “HF+alcohol” may be used as an etchant gas for removing a silicon oxide film by a dry etching process with a high selectivity in a non-plasma manner while the silicon oxide film is exposed on a wafer together with another material (e.g., a silicon nitride film). In this case, an effective alcohol that may be employed together with HF may be screened. To this end, input data of alcohols may be obtained from a database of the alcohols in process P10 of FIG. 5. Input data of the alcohols may include primary alcohol, secondary alcohol, and/or tertiary alcohol. In some implementations, the input data of the alcohols may include alcohol represented by R—OH, wherein R denotes C1-C15 linear or branched alkyl, C1-C15 linear or branched alkenyl, C1-C15 linear or branched alkynyl, C2-C15 linear or branched alkyl substituted with at least one fluorine atom, phenyl, phenyl substituted with at least one C1-C3 alkyl group, phenyl substituted with at least one chlorine atom or fluorine atom, or benzyl), without being limited thereto In some implementations, the input data of the alcohols may include methanol, ethanol, propanol, isopropanol, butanol, isobutanol, t-butanol, 2-methoxyethanol, 1-methoxy-2-propanol, 3-methoxy-1-butanol, pentanol, hexanol, 2-ethyl-1-hexanol, heptanol, octanol, ethylene glycol, propylene glycol, butylene glycol, hexylene glycol, tetrahydrofurfuryl alcohol, 1,2-butanediol and 1,4-butanediol, etc, without being limited thereto.

The organic amines or the alcohols screened in process P10 of FIG. 5 may be represented as character strings in a simple one-dimensional format. The character strings in the one-dimensional format may be represented as a Simplified Molecular Input Line Entry System (SMLES) code, a Simple Molecular ARchives for Target System (SMARTS) code, an IUPAC International Chemical Identifier (InChi) code, or the like.

FIG. 6B is a table showing examples of character strings in a one-dimensional format (a SMILES code and an InChi code) for the organic amines screened in process P10 of FIG. 5.

In process P20 of FIG. 5, a plurality of descriptors based on the etching mechanism may be estimated. For example, the plurality of descriptors may include (1) a fluorination reaction activation energy Ea(SiO₂) of a silicon oxide film, (2) a difference ΔEa [ΔEa=Ea(SiNₓ)−Ea(SiO₂)] between a fluorination reaction activation energy Ea(SiNₓ) of a silicon nitride film and the fluorination reaction activation energy Ea(SiO₂) of the silicon oxide film, and (3) a [(NR₃H)₂SiF₆] salt's binding energy, which is an intermediate product estimated to be generated during an etching process. In some implementations, the [(NR₃H)₂SiF₆] salt's binding energy may be a value estimated based on first-principles simulations.

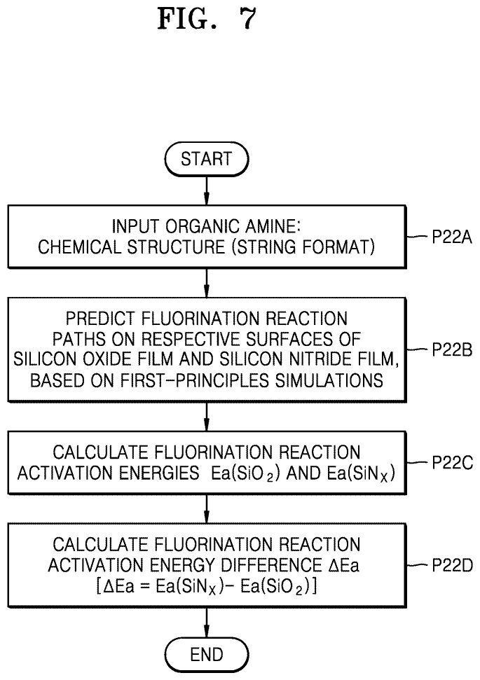

FIG. 7 is a flowchart illustrating a process of estimating descriptors according to process P20 of FIG. 5, according to some implementations of the present disclosure. FIG. 7 illustrates a process of predicting a fluorination reaction activation energy Ea(SiO₂) of a silicon oxide film and a difference ΔEa between Ea(SiNₓ) and Ea(SiO₂), which are descriptors related to a fluorination reaction that occurs during an etching process.

In process P22A of FIG. 7, a chemical structure of each organic amine to be evaluated may be input in the form of character strings of a one-dimensional format (e.g., a SMILES code) into the program 152 of the storage 150 included in the etching gas evaluation device 100 illustrated in FIG. 1.

In process P22B of FIG. 7, based on first-principles simulations, fluorination reaction paths of an “HF+organic amine” etchant gas mixture on respective surfaces of a silicon oxide film and a silicon nitride film may be predicted. Herein, a fluorination reaction of the “HF+organic amine” etchant gas mixture on the surface of each of the silicon oxide film and the silicon nitride film may be predicted to follow a path of Reaction scheme 1 described above.

In some implementations, a process of predicting the fluorination reaction paths of the “HF+organic amine” etchant gas mixture according to process P22B of FIG. 7 may be performed by using the program 152 of the storage 150 included in the etchant gas evaluation device 100 illustrated in FIG. 1.

In process P22C of FIG. 7, a fluorination reaction activation energy Ea(SiO₂) on a surface of the silicon oxide film and a fluorination reaction activation energy Ea(SiNₓ) on a surface of the silicon nitride film may be calculated.

In some implementations, Ea(SiO₂) and Ea(SiNₓ) may be calculated by Equation 1:

$$\begin{matrix}Ea ( {SiO}_{2} ) = E \# ( {SiO}_{2} - HF - {NR}_{3} ) - E ( {SiO}_{2} - HF - {NR}_{3} ) \\ Ea ( {SiN}_{x} ) = E \# ( {SiO}_{x} - HF - {NR}_{3} ) - E ( {SiO}_{x} - HF - {NR}_{3} )\end{matrix} \qquad [ Equation 1 ]$$

wherein E #(SiO₂—HF—NR₃) denotes an energy corresponding to a transition state of a mixture identified in a fluorination reaction path of a silicon oxide film exposed to an etching atmosphere including “HF+organic amine,” E #(SiNₓ—HF—NR₃) denotes an energy corresponding to a transition state of a mixture identified in a fluorination reaction path of a silicon nitride film exposed to the etching atmosphere, E (SiO₂—HF—NR₃) denotes a total energy of a mixture including HF and NR₃, which is adsorbed on the silicon oxide film, and E (SiNₓ—HF—NR₃) denotes a total energy of a mixture including HF and NR₃, which is adsorbed on the silicon nitride film.

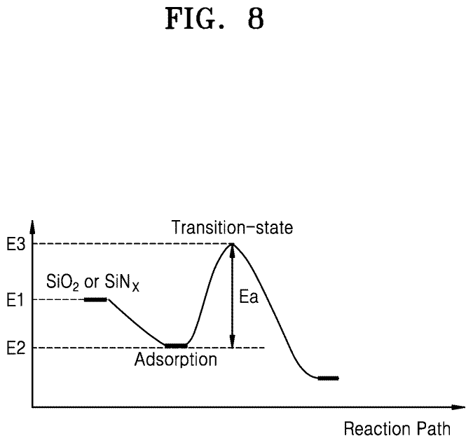

FIG. 8 is a graph showing an example of energy variation with respect to a reaction path of a silicon oxide film or a silicon nitride film when the silicon oxide film or the silicon nitride film is exposed to a mixture of HF gas and an organic amine gas.

FIG. 8 conceptually illustrates a first energy E1 in an initial reaction state of the silicon oxide film or the silicon nitride film, a second energy E2 in a state in which the etchant mixture is adsorbed on the silicon oxide film or the silicon nitride film in a fluorination reaction path caused by the etching atmosphere, and a third energy E3 in a transition state of the silicon oxide film or the silicon nitride film, when the silicon oxide film or silicon nitride film is exposed to an etching atmosphere including an etchant gas mixture including HF gas and an organic amine gas. An activation energy Ea [e.g., Ea(SiO₂) or Ea(SiNₓ)] may be defined by a difference between the third energy E3 and the second energy E2.

In process P22C of FIG. 7, energies may be calculated from Equation 1, based on first-principles simulations. In some implementations, the first-principles simulations may be calculated based on molecular orbital theory-based methods, such as a Hartree-Fock method, a semi-empirical quantum chemistry method, a Møller-Plesset perturbation method, a coupled cluster method, and/or a quantum Monte Carlo method. In some implementations, the first-principles simulations may be calculated based on density functional theory-based methods, such as a Thomas-Fermi model method, an orbital-free density functional theory method, a linearized augmented plane-wave method, and/or a projected augmented wave method.

In process P22D of FIG. 7, according to the following Equation 2, a fluorination reaction activation energy difference ΔEa may be calculated from a fluorination reaction activation energy Ea(SiO₂) of the silicon oxide film and a fluorination reaction activation energy Ea(SiNₓ) of the silicon nitride film.

$$Δ ( Ea = Ea ( {SiN}_{x} ) - Ea ( {SiO}_{2} ) \qquad [ Equation 2 ]$$

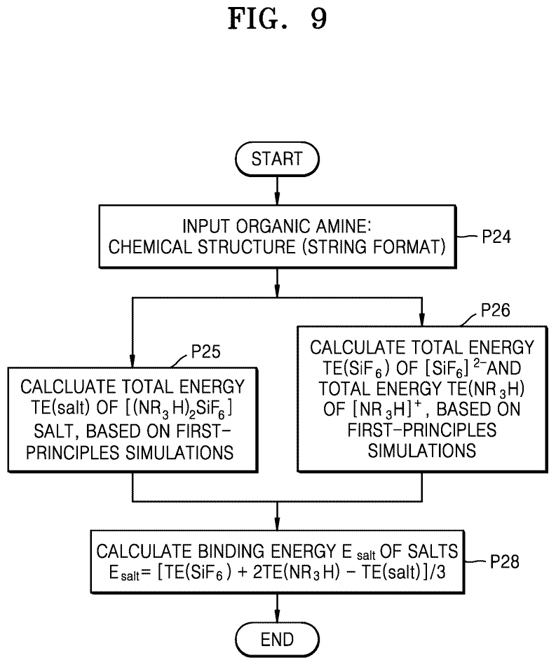

FIG. 9 is a flowchart illustrating an example of a process of estimating descriptors according to process P20 of FIG. 5. FIG. 9 illustrates a process of predicting a salt's binding energy Eₛₐₗₜ of an organic ammonium fluorosilicate [(NR₃H)₂SiF₆] salt, which is another descriptor related to a fluorination reaction.

In process P24 of FIG. 9, similar to process P22A of FIG. 7, a chemical structure of each organic amine to be evaluated may be input in the form of character strings of a one-dimensional format (e.g., a SMILES code) into the program 152 of the storage 150 included in the etching gas evaluation device 100 illustrated in FIG. 1.

In process P25 of FIG. 9, a total energy TE(salt) of the [(NR₃H)₂SiF₆] salt may be calculated from a crystal structure of the [(NR₃H)₂SiF₆] salt, based on first-principles simulations.

In process P26 of FIG. 9, a total energy TE(SiF₆) of [SiF₆]²⁻ and a total energy TE(NR₃H) of [NR₃H]⁺ may be calculated, based on the first-principles simulations.

In some implementations, to perform process P25 and process P26 of FIG. 9, the first-principles simulations may be computed based on molecular orbital theory-based methods, such as Hartree-Fock methods, semi-empirical quantum chemistry methods, Møller-Plesset perturbation methods, coupled cluster methods, and/or quantum Monte Carlo. In other embodiments, the first-principles simulations may be computed based on density functional theory-based methods, such as Thomas-Fermi Model methods, orbital-free density functional theory methods, linearized augmented plane-wave methods, and/or projected augmented wave methods.

In process P28 of FIG. 9, a salt's binding energy Eₛₐₗₜ, which is intermediate products produced by an etching reaction, may be calculated as a descriptor. In some implementations, the salt's binding energy Eₛₐₗₜ may be calculated as shown in Equation 3:

$${E}_{salt} = [ TE ( {SiF}_{6} ) + 2 TE ( {NR}_{3} H ) - TE ( salt ) ] / 3 \qquad [ Equation 3 ]$$

Referring back to FIG. 5, in process P30, an etch rate of a silicon oxide film may be calculated. The process of calculating the etch rate of the silicon oxide film according to process P30 may be performed based on a physical model A (PM-A). As used herein, the physical model A may be referred to as a first physical model.

FIG. 10 is a flowchart illustrating an example of a process of calculating an etch rate of a silicon oxide film according to process P30 of FIG. 5, based on a physical model A (PM-A).

In process P30A of FIG. 10, a fluorination reaction activation energy Ea(SiO₂) of a silicon oxide film, a [(NR₃H)₂SiF₆] salt's binding energy, and data about an etching temperature may be input into the program 152 of the storage 150 of the etchant gas evaluation device 100 illustrated in FIG. 1.

In process P30B of FIG. 10, the physical model A (PM-A) may be calculated. A process of calculating the physical model A (PM-A) according to process P30B may be performed on, or based on, the estimation results of at least one of the graphs illustrated in FIGS. 4A and 4B in the program 152 of the storage 150 of the etchant gas evaluation device 100 illustrated in FIG. 1.

In process P30C of FIG. 10, based on the physical model A (PM-A) derived in process P30B, the etch rate of the silicon oxide film may be calculated in the program 152 of the storage 150 of the etchant gas evaluation device 100 illustrated in FIG. 1.

In some implementations, the process of calculating the etch rate of the silicon oxide film according to process P30C of FIG. 10 may be determined by a first etch rate of the silicon oxide film that is obtained when the silicon oxide film is etched by using organic amine gases at a first temperature, which is an etching temperature of lower than 100° C. (e.g., 60° C.), a second etch rate of the silicon oxide film that is obtained when the silicon oxide film is etched by using organic amine gases at a second temperature, which is an etching temperature of 100° C. or higher (e.g., 120° C.), or a combination thereof.

Referring back to FIG. 5, in process P40, an etching selectivity of a silicon oxide film with respect to the silicon nitride film may be calculated. The process of calculating the etching selectivity of the silicon oxide film with respect to the silicon nitride film according to process P40 may be performed based on a physical model B (PM-B). As used herein, the physical model B may be referred to as a second physical model.

FIG. 11 is a flowchart illustrating an example of a process of calculating an etching selectivity of a silicon oxide film with respect to a silicon nitride film, according to process P40, based on a physical model B (PM-B).

In process P40A of FIG. 11, a fluorination reaction activation energy difference ΔEa [ΔEa=Ea(SiNₓ)−Ea(SiO₂)], a [(NR₃H)₂SiF₆] salt's binding energy, and data about an etching temperature may be input into the program 152 of the storage 150 of the etchant gas evaluation device 100 illustrated in FIG. 1.

In process P40B of FIG. 11, the physical model B (PM-B) may be derived. A process of calculating the physical model B (PM-B) according to process P40B may be performed on, or based on, the estimation results of the graphs illustrated in FIGS. 4C and 4D in the program 152 of the storage 150 of the etchant gas evaluation device 100 illustrated in FIG. 1.

In process P40C of FIG. 11, based on the physical model B (PM-B) derived in process P40B, the etching selectivity of the silicon oxide film with respect to the silicon nitride film may be calculated in the program 152 of the storage 150 of the etchant gas evaluation device 100 illustrated in FIG. 1.

In some implementations, the process of calculating the etching selectivity of the silicon oxide film with respect to the silicon nitride film according to process P40C of FIG. 11 may be determined by a first etching selectivity of the silicon oxide film with respect to the silicon nitride film that is obtained when the silicon oxide film is etched by using organic amines gases at a first temperature, which is an etching temperature of lower than 100° C. (e.g., 60° C.), a second etching selectivity of the silicon oxide film with respect to the silicon nitride film that is obtained when the silicon oxide film is etched by using organic amine gases at a second temperature, which is an etching temperature of 100° C. or higher (e.g., 120° C.), or a combination thereof.

Referring back to FIG. 5, in process P50, organic amines to be evaluated may be verified.

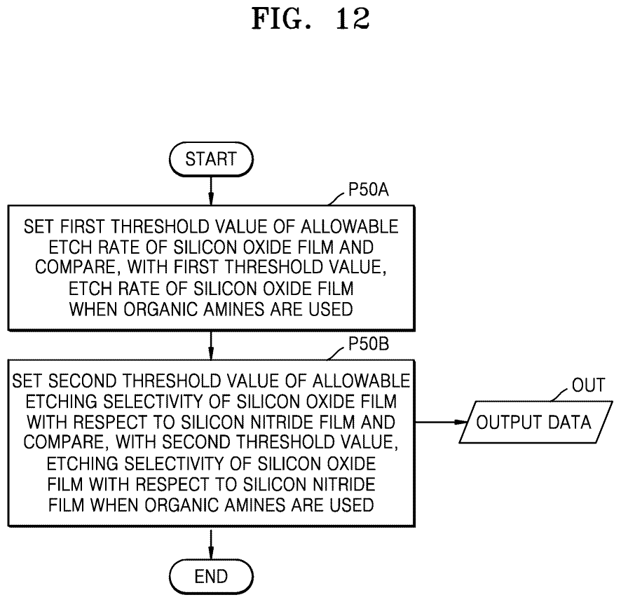

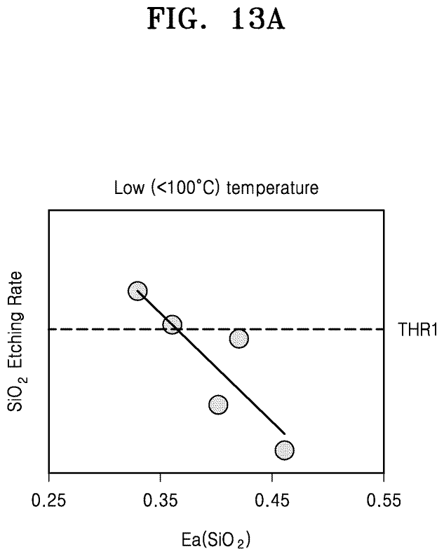

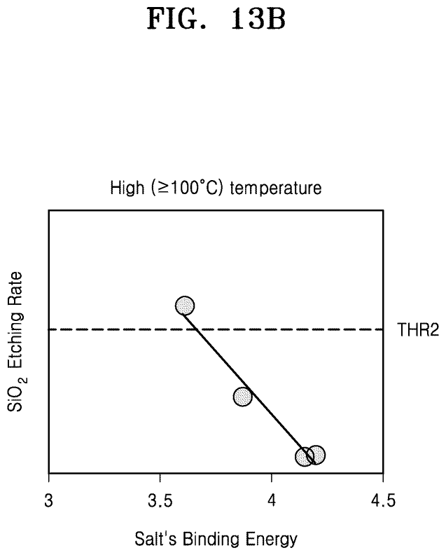

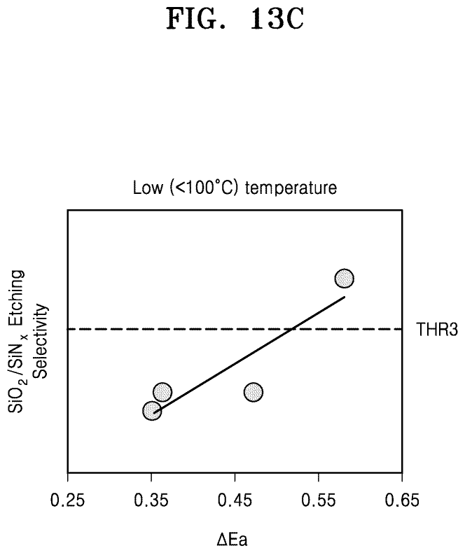

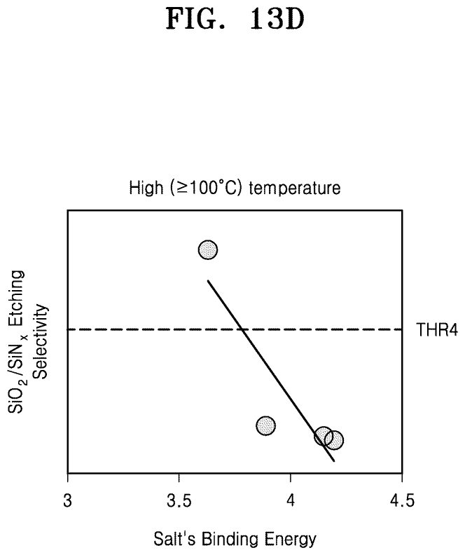

FIG. 12 is a flowchart illustrating an example of a process of verifying organic amines to be evaluated, according to process P50 of FIG. 5. FIGS. 13A, 13B, 13C, and 13D are graphs showing specific examples of a process of verifying organic amines to be evaluated, by using a physical model including descriptors based on the etching mechanism.

In process P50A of FIG. 12, a first threshold value of an allowable etch rate of a silicon oxide film may be set, and an etch rate of the silicon oxide film obtained when organic amines to be evaluated are used may be compared with the first threshold value.

For example, in process P50A of FIG. 12, at least one of a process of comparing the estimation result of FIG. 4A with a threshold value THR1 set to an allowable etch rate as illustrated in FIG. 13A and a process of comparing the estimation result of FIG. 4B with a threshold value THR2 set to an allowable etch rate as illustrated in FIG. 13B may be performed.

In process P50B of FIG. 12, a second threshold value of an allowable etching selectivity of a silicon oxide film with respect to the silicon nitride film may be set, and an etching selectivity of the silicon oxide film with respect to the silicon nitride film, which is obtained when organic amines to be evaluated are used, may be compared with the second threshold value.

For example, in process P50B of FIG. 12, at least one of a process of comparing the estimation result of FIG. 4C with a threshold value THR3 set to an allowable etching selectivity as illustrated in FIG. 13C and a process of comparing the estimation result of FIG. 4D with a threshold value THR4 set to an allowable etching selectivity as illustrated in FIG. 13D may be performed.

Comparison results obtained from each of process P50A and process P50B of FIG. 12 may be output as output data OUT.

Referring back to FIG. 5, in process P60, an optimal organic amine may be determined based on the verification results obtained in process P50.

In some implementations, to determine the optimal organic amine according to process P60 of FIG. 5, the optimal organic amine may be determined within a range of values higher than the threshold values THR1, THR2, THR3, and THR4, which are respectively set in the estimation results of FIGS. 4A, 4B, 4C, and 4D. For example, from among organic amine gases to be evaluated, an organic amine gas corresponding to a case in which the etch rate of the silicon oxide film has a value higher than the threshold values THR1 and THR2 set in at least one of the estimation results of FIGS. 4A and 4B and a case in which the etching selectivity of the silicon oxide film with respect to the silicon nitride film has a value higher than the threshold values THR3 and THR4 set in at least one of the estimation results of FIGS. 4C and 4D may be determined as the optimal organic amine.

The method of evaluating the etchant gas described above with reference to FIGS. 3 to 13D pertains to a specific example in which the etchant gas including the mixture of the HF gas and the organic amine gas is employed in the etching and/or cleaning processes of removing the silicon oxide film with a high selectivity while the silicon oxide film is exposed together with the silicon nitride film. However, the scope of this disclosure is not limited thereto. For example, an etching target film and a protection target film are not limited to the silicon oxide film and the silicon nitride film, respectively, and various other material(s) can be used for the etching target film and/or the protection target film. In addition, examples in which an etchant gas that forms an etchant gas mixture with HF gas is an organic amine have been described in detail; however, the etchant gas is not limited thereto. For example, when alcohols are used instead of the organic amines as an etchant gas to form an etchant gas mixture with HF gas, an optimal alcohol may be determined by verifying target alcohols by using a method similar to that described above with reference to FIGS. 3 to 13D.

Accordingly, in order to screen an etchant gas for dry etching an etching target film with a high selectivity in a non-plasma manner while the etching target film (e.g., a silicon oxide film) is exposed on a wafer together with a protection target film (e.g., a silicon nitride film), an etch rate of the etching target film and an etching selectivity of the etching target film with respect to the protection target film, which are obtained when a target etchant gas is used, may be calculated by using physical models. Descriptors may be used as the physical models. The descriptors may be respectively determined by an activation energy difference between etchant reactants, which is calculated based on first-principle simulations, and a salt's binding energy of an intermediate product produced by a reaction of the etching reactants. By the method described above, an etchant gas for dry etching or dry cleaning the etching target film with a high selectivity in the non-plasma manner while the etching target film is exposed on the wafer together with the protection target film may be efficiently and accurately screened.

Furthermore, when an etchant gas including a mixture of HF gas and an organic amine gas is used in etching and/or cleaning processes of removing a silicon oxide film with a high selectivity while the silicon oxide film is exposed together with a silicon nitride film, in order to screen an effective organic amine that may be employed together with the HF gas, an etch rate of the silicon oxide film and an etching selectivity of the silicon oxide film with respect to the silicon nitride film when each of organic amines is used may be calculated by using physical models. Descriptors may be used as the physical models. The descriptors may be respectively determined by, based on, or as a “fluorination reaction activation energy difference” and a “binding energy of an organic ammonium fluorosilicate salt,” which are calculated based on first-principles simulations. By the method described above, an effective organic amine, which may be employed together with HF gas in an etchant gas for dry etching or dry cleaning the silicon oxide film with a high selectivity in the non-plasma manner while the silicon oxide film is exposed on a wafer together with another material (e.g., the silicon nitride film), may be efficiently and accurately selected.

It will be understood that various further operations can be performed based on selection of the etchant gas. For example, based on selection of the etchant gas, the selected etchant gas can be delivered into a chamber in which the etching target film and the protection target film are exposed on a wafer. A dry etching process can be performed on the etching target film in the chamber using the supplied etchant gas. The dry etching process can include any of the dry etching processes discussed above, e.g., a process of plasma etching a self-aligned contact, a process of forming an air gap, a cleaning process or a process of selectively etching only a silicon oxide film, from among a silicon nitride film and the silicon oxide film, in a charge trap layer included in a VNAND device.

While this disclosure contains many specific implementation details, these should not be construed as limitations on the scope of what may be claimed. Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a combination can in some cases be excised from the combination, and the combination may be directed to a subcombination or variation of a subcombination.

While certain examples have been particularly shown and described, it will be understood that various changes in form and details may be made therein without departing from the spirit and scope of this disclosure.

What is claimed is:

1. A processor-implemented method of evaluating etchant gases for etching an etching target film while the etching target film and a protection target film are exposed to an etching atmosphere, the method comprising:
obtaining input data characterizing a plurality of etchant gases;
determining, based on the input data, a plurality of descriptors, wherein the plurality of descriptors comprise, for each of the plurality of etchant gases, a first descriptor, a second descriptor, and a third descriptor,
wherein the first descriptor is based on an activation energy of the etching target film,
wherein the second descriptor is based on a difference between an activation energy of the protection target film and the activation energy of the etching target film, and
wherein the third descriptor is based on a binding energy of an intermediate product generated during etching of the etching target film using the etchant gas;
determining an etch rate of the etching target film for each of the plurality of etchant gases, based on a first physical model that is based on the first descriptor for the etchant gas, the third descriptor for the etchant gas, and an etching temperature;
determining an etching selectivity of the etching target film with respect to the protection target film for each of the plurality of etchant gases, based on a second physical model that is based on the second descriptor for the etchant gas, the third descriptor for the etchant gas, and the etching temperature; and
selecting a first etchant gas of the plurality of etchant gases based on the etch rates for the plurality of etchant gases and the etching selectivities for the plurality of etchant gases.

2. The method of claim 1, wherein the etching atmosphere comprises hydrogen fluoride (HF) gas, and,
wherein the intermediate product comprises a compound including fluorine atoms.

3. The method of claim 1, wherein the etching target film comprises a silicon oxide film, and the protection target film comprises a silicon nitride film.

4. The method of claim 1, wherein each of the plurality of etchant gases comprises an organic amine or an alcohol.

5. The method of claim 1, wherein determining the plurality of descriptors comprises, for each of the plurality of etchant gases:
obtaining data characterizing a chemical structure of each of the plurality of etchant gases;
predicting, based on first-principles simulations:
a first reaction path of a gas mixture comprising the etchant gas on a surface of the etching target film, and
a second reaction path of the gas mixture on a surface of the protection target film;
determining a first reaction activation energy on the surface of the etching target film based on the first reaction path, and a second reaction activation energy on the surface of the protection target film based on the second reaction path; and
determining a difference between the second reaction activation energy and the first reaction activation energy.

6. The method of claim 1, wherein determining the third descriptor for each of the plurality of etchant gases comprises determining the binding energy of the intermediate product from a crystal structure of the intermediate product, based on first-principles simulations.

7. The method of claim 1, wherein, in determining the etch rate of the etching target film for each of the plurality of etchant gases, the first physical model is derived from a fluorination reaction activation energy of the etching target film, the binding energy of the intermediate product generated during etching of the etching target film using the etchant gas, and the etching temperature.

8. The method of claim 1, wherein, in determining the etching selectivity of the etching target film with respect to the protection target film for each of the plurality of etchant gases, the second physical model is derived from a fluorination reaction activation energy difference, the binding energy of the intermediate product generated during etching of the etching target film using the etchant gas, and the etching temperature, and
wherein the fluorination reaction activation energy difference is based on a difference between a fluorination reaction activation energy of the etching target film and a fluorination reaction activation energy of the protection target film.

9. The method of claim 1, wherein selecting the first etchant gas comprises:
comparing the etch rate of the etching target film for each of the plurality of etchant gases with a threshold etch rate; and
comparing the etching selectivity of the etching target film for each of the plurality of etchant gases with a threshold etching selectivity; and
selecting, as the first etchant gas, an etchant gas having an etch rate greater than the threshold etch rate and an etching selectivity greater than the threshold etching selectivity.

10. A processor-implemented method of evaluating organic amine gases for etching an etching target film by using an etchant gas comprising a mixture of hydrogen fluoride (HF) gas and an organic amine gas while the etching target film and a protection target film are exposed to an etching atmosphere, the method comprising:
obtaining input data characterizing a plurality of organic amine gases;
determining, based on the input data, a plurality of descriptors, wherein the plurality of descriptors comprise, for each of the plurality of organic amine gases, a first descriptor, a second descriptor, and a third descriptor,
wherein the first descriptor is based on an activation energy of the etching target film,
wherein the second descriptor is based on a difference between an activation energy of the protection target film and the activation energy of the etching target film, and
wherein the third descriptor is based on a binding energy of an organic ammonium salt including fluorine atoms, wherein the organic ammonium salt is generated during etching of the etching target film using the organic amine gas;
determining an etch rate of the etching target film for each of the plurality of organic amine gases, based on a first physical model that is based on the first descriptor for the organic amine gas, the third descriptor for the organic amine gas, and an etching temperature;
determining an etching selectivity of the etching target film with respect to the protection target film for each of the plurality of organic amine gases, based on a second physical model that is based on the second descriptor for the organic amine gas, the third descriptor for the organic amine gas, and the etching temperature; and
selecting a first organic amine gas of the plurality of organic amine gases based on the etch rates for the plurality of organic amine gases and the etching selectivities for the plurality of organic amine gases.

11. The method of claim 10, wherein etching the etching target film is performed in a non-plasma manner.

12. The method of claim 10, wherein the etching target film comprises a silicon oxide film,
wherein the protection target film comprises a silicon nitride film, and,
wherein the organic ammonium salt is an organic ammonium fluorosilicate salt.

13. The method of claim 10, wherein the etching target film comprises a silicon oxide film, and the protection target film comprises a silicon nitride film, and
wherein determining the plurality of descriptors comprises, for each of the plurality of organic amine gases:
obtaining data characterizing a chemical structure of each of the plurality of organic amine gases;
predicting, based on first-principles simulations:
first reaction path of a gas mixture comprising the organic amine gas on a surface of the silicon oxide film, and
a second reaction path of the gas mixture on a surface of the silicon nitride film;
determining a first reaction activation energy on the surface of the silicon oxide film based on the first reaction path, and a second reaction activation energy on the surface of the silicon nitride film based on the second reaction path; and
determining a difference between the second reaction activation energy and the first reaction activation energy.

14. The method of claim 10, determining the third descriptor for each of the plurality of organic amine gases comprises determining the binding energy of the organic ammonium salt from a crystal structure of the organic ammonium salt, based on first-principles simulations.

15. The method of claim 10, wherein the etching target film comprises a silicon oxide film,
wherein the protection target film comprises a silicon nitride film, and
wherein, in determining the plurality of descriptors, the first descriptor for each of the plurality of organic amine gases is determined based on:
a first activation energy of the silicon oxide film, corresponding to etching of the silicon oxide film using the organic amine gas at a first temperature lower than 100° C.,
a second activation energy of the silicon oxide film, corresponding to etching of the silicon oxide film using the organic amine gas at a second temperature of 100° C. or higher, or
a combination of the first activation energy and the second activation energy.

16. The method of claim 10, wherein the etching target film comprises a silicon oxide film,
wherein the protection target film comprises a silicon nitride film, and,
wherein, in determining the plurality of descriptors, the second descriptor for each of the plurality of organic amine gases is determined based on:
a first difference between an activation energy of the silicon nitride film and an activation energy of the silicon oxide film, corresponding to etching of the silicon oxide film using the organic amine gas at a first temperature lower than 100° C.,
a second difference between an activation energy of the silicon nitride film and an activation energy of the silicon oxide film, corresponding to etching of the silicon oxide film using the organic amine gases at a second temperature of 100° C. or higher, or
a combination of the first difference and the second difference.

17. The method of claim 10, wherein the etching target film comprises a silicon oxide film, wherein the protection target film comprises a silicon nitride film, and
wherein selecting the first organic amine gas comprises:
comparing the etch rate of the etching target film for each of the plurality of organic amine gases with a threshold etch rate; and
comparing the etching selectivity of the etching target film for each of the plurality of organic amine gases with a threshold etching selectivity.

18. The method of claim 17, wherein selecting the first organic amine gas comprises:
selecting, as the first organic amine gas, an organic amine gas having an etch rate greater than the threshold etch rate and an etching selectivity greater than the threshold etching selectivity.

19. A method of evaluating organic amine gases for etching a silicon oxide film in a non-plasma manner by using an etchant gas comprising a mixture of HF gas and an organic amine gas while the silicon oxide film and a silicon nitride film are exposed to an etching atmosphere, the method comprising:
obtaining input data characterizing a plurality of organic amine gases;
for each of the plurality of organic amine gases, based on the input data:
predicting a first reaction path for a gas mixture comprising the organic amine gas on a surface of the silicon oxide film and a second reaction path for the gas mixture on a surface of the silicon nitride film,
determining a first fluorination reaction activation energy on the surface of the silicon oxide film,
determining a second fluorination reaction activation energy on the surface of the silicon nitride film,
determining a difference between the second fluorination reaction activation energy and the first fluorination reaction activation energy, and
determining a binding energy of an organic ammonium fluorosilicate salt generated during etching of the silicon oxide film using the organic amine gas;
determining an etch rate of the silicon oxide film for each of the plurality of organic amine gases, based on a first physical model that is based on the first fluorination reaction activation energy for the organic amine gas and the binding energy of the organic ammonium fluorosilicate salt for the organic amine gas;
determining an etching selectivity of the silicon oxide film with respect to the silicon nitride film for each of the plurality of organic amine gases, based on a second physical model that is based on a difference between the second fluorination reaction activation energy for the organic amine gas and the first fluorination reaction activation energy for the organic amine gas, and based on the binding energy of the organic ammonium fluorosilicate salt for the organic amine gas; and
selecting a first organic amine gas of the plurality of organic amine gases based on the etch rates for the plurality of organic amine gases and the etching selectivities for the plurality of organic amine gases.

20. The method of claim 19, wherein, in determining the etch rate of the silicon oxide film for each of the plurality of organic amine gases, the organic ammonium fluorosilicate salt comprises [(NR₃H)₂SiF₆] salt, and the etch rate is determined based on an etching temperature, and
wherein, in determining the etching selectivity of the silicon oxide film with respect to the silicon nitride film for each of the plurality of organic amine gases, the organic ammonium fluorosilicate salt comprises [(NR₃H)₂SiF₆] salt, and the etching selectivity is determined based on the etching temperature.
